(19) United States
(12) Patent Application Publication
(10) Pub. No.: US 2017/0209188 A1
(43) Pub. Date: Jul. 27, 2017

(54) RODLESS BIVERTEBRAL TRANSPEDICULAR FIXATION WITH INTERBODY FUSION

(71) Applicants: Gerald Schell, Bay City, MI (US); Jeffrey R. Schell, Denver, CO (US)

(72) Inventors: Gerald Schell, Bay City, MI (US); Jeffrey R. Schell, Denver, CO (US)

(21) Appl. No.: 15/416,983

(22) Filed: Jan. 26, 2017

Related U.S. Application Data
(60) Provisional application No. 62/287,363, filed on Jan. 26, 2016.

(51) Int. Cl. A61B 17/70; A61F 2/44; A61B 17/88

(52) U.S. Cl. CPC A61B 17/7097; A61B 17/88; A61B 17/8897; A61F 2/4455; A61B 2017/90

(57) ABSTRACT

Embodiments disclosed herein pertain to a process that enables potential fusion of adjacent vertebrae by a novel technique. The process disclosed substantially reduces the amount of hardware required for vertebral fusion, reduces potential soft tissue trauma, and minimizes potential for nerve contact. The novel process described herein leverages existing vertebral structural properties in such a way as to allow vertebral fusion to occur exclusively in the spinal column without external support to address spinal deformities. The novel, minimally invasive procedure makes it possible to both adjust vertebrae with respect to each other and makes it possible to provide structural support that allows vertebral bodies to potentially fuse.

### CROSS REFERENCE TO RELATED APPLICATIONS

This application claims benefit to provisional patent application No. 62/287,363, entitled “Rodless Bivertebral Transpedicular Fixation With Interbody Fusion”, filed Jan. 26, 2016, which is incorporated by reference in its entirety for all purposes.

### BACKGROUND OF THE INVENTION

The human spine intrinsically has a normal degree of curvature. This curvature positions the head above the spine in such a way as to provide shock absorption. The flexibility of this curved orientation provides increased absorption ability related to vertical loads than a rigid, vertically oriented spine would be. A proper degree of curvature throughout the spine is critical to absorb the load placed on the spine.

Spinal deformities such as kyphosis, lordosis, scoliosis, and spondylolisthesis affect the alignment of vertebrae relative to one another. When vertebrae are not aligned properly, they are unable to bear loads without potential for pain or injury. Even slight movement of the spine when vertebrae are misaligned can be painful for affected individuals, and may lead to limited movement and physical activity. As a result, misalignment of adjacent vertebrae may subsequently drive other health problems. These types of spinal misalignments may be a result of birth defect, injury, growth abnormalities (particularly during adolescence), or manifest in old age as deterioration of body tissues results in unequal forces on the spinal column.

Kyphosis is an example of a spinal misalignment in which the spinal column has an exaggerated outward curvature, typically resulting in a rounded back. More severe cases can worsen, and may cause impingement of the spinal cord. Impingement of the spinal cord may cause a number of consequences such as weakness, loss of sensation, loss of bowel or bladder control, chest pain, and shortness of breath. Extreme cases can even lead to pulmonary or cardiac failure. Typically, kyphosis is characterized by hunching, pain, fatigue, and stiffness in the back.

Kyphosis is categorized by three types: postural, Scheuermann's, and congenital. Postural kyphosis is less severe, and does not usually require surgery. Scheuermann's kyphosis is defined by angular separation between vertebral end-plates of at least 5-degrees from parallel with a larger separation at the posterior side of the vertebrae. To be considered Scheuermann's kyphosis, such angular separation must be present in at least three neighboring vertebral bodies. Congenital kyphosis is the result of developmental abnormalities, and may result in the fusion of several vertebral bodies. For congenital cases and more severe cases of Scheuermann's kyphosis, surgery is often recommended by doctors.

In addition to developmental abnormalities or injury, kyphosis can be caused by other diseases, including osteoporosis, infectious disease, tumors, ankylosing spondylitis, and degenerative arthritis. In some cases, kyphosis can also be caused by normal wear and tear. This form, called degenerative kyphosis, can result in a collapsed disc. A collapsed disc is an intervertebral disc that has lost its normal height due to deterioration of its fibrous outer wall typically caused by traumatic injury or wear and tear as a regular part of the aging process.

After a disc collapses, kyphosis often gets worse, as an imbalance of forces exacerbates the problem.

Kyphosis can often result from invasive spinal surgeries, which typically happens when the operative area does not heal as intended. This can happen, for example, in traditional spinal fusions. An unstable fusion may cause a disc space to collapse and develop kyphosis. This condition can often require a second surgery to correct the problem.

Lordosis is a curvature in the backward direction, opposite of kyphosis. A small amount of curvature in the lower back is normal, but excessive curving can cause major structural problems for the human body. The cause of this abnormal curvature is unknown, but may be associated with poor posture, vertebral problems, neuromuscular problems, back surgery, or hip problems. Lordosis may also be present from birth.

The symptoms of lordosis can vary widely, but may include a weaker spine with reduced shock absorption, which can cause problems over time if left untreated. More severe cases can also cause chronic lower back pain. Lordosis affects persons of all ages, and may be affected by contributing factors, such as achondroplasia, discitis, kyphosis, obesity, osteoporosis, and spondylolisthesis. Lordosis may appear early in life, and may be evident by an excessively protruding buttocks.

Lordosis may correct itself, particularly in the case of children. If, after monitoring, lordosis does not correct itself, a doctor may recommend braces to control the growth trajectory, reducing excess body weight, physical therapy to build strength and flexibility, and ultimately drugs to relieve pain and swelling. In the event that these treatments do not correct the problem, or that the curvature is severe enough, spinal surgery may be recommended. These may include structural instrumentation, artificial disc replacements, and/or kyphoplasty.

Scoliosis is a lateral curvature of the spine. While some degree of kyphotic (forward) and lordotic (backward) curvature is normal, scoliosis is always abnormal. Except where scoliosis is caused by other diseases that may be present, such as cerebral palsy or muscular dystrophy, the cause of scoliosis is unknown.

Scoliosis usually manifests before puberty, most often during the growth spurt that occurs just before puberty. Scoliosis is usually mild, but can grow more severe over time in some cases. In these cases, scoliosis can be disabling, and can even reduce the amount of space in the thoracic cavity, making it difficult to breathe.

Scoliosis is typically monitored by X-ray analysis over time. In cases of mild scoliosis that do not worsen, no treatment may be required. In other cases, braces may be required to prevent the curvature from worsening. Other cases may require surgery to prevent the curvature from worsening or straighten the spine.

Spondylolisthesis is a condition in which one vertebral body in the spinal column slides forward relative to an adjacent vertebral body. This displacement negatively affects or eliminates the load-bearing ability of the spine. Rather than a single, contiguous spinal column capable of enduring a vertical force, the load bearing properties are substantially attenuated when two vertebral bodies are not situated in such a way as to support one another and translate the force from one to the next.

Spondylolisthesis can be among the more severe spinal deformations. This is because the vertebral slippage can pinch the spinal nerves, causing back or buttock pain, numbness or weakness in one or both legs, and difficulty walking. It can also cause loss of bladder and bowel control. In some cases, no evidence of vertebral slippage may be immediately apparent. In these cases, many years may pass before inexplicable pain or weakness, or even a limp, may manifest.

Spondylolisthesis is caused by problems in the many small joints that keep the spine appropriately aligned. These problems may include congenital birth defects, accident or trauma, overuse, leading to a stress fracture, or damage caused by infection or arthritis. For these reasons, spondylolisthesis can particularly affect older adults, as normal wear and tear can lead to stress fractures, or in the event that a disc slips out of place. Young individuals heavily involved in sports may also be at risk. Certain sports in particular, such as gymnastics and weight lifting, can lead to overuse of the vertebrae and may put individuals at a higher risk.

Spondylolisthesis is also among the more difficult conditions to treat. Surgeries to correct the misalignment and relieve pressure on the spinal nerves often requires both a decompression—in which the two vertebral bodies are distracted from one another—and fusion—in which one vertebrae is fused to the next to keep them in the appropriate position.

Combinations of multiple deformations are also possible, such as kyphoscoliosis. These spinal deformations present with abnormal curvature in two or more planes, such as the coronal and sagittal plane in kyphoscoliosis. Such combinations can be difficult to treat using conventional surgeries adapted to single dimension deformities, and may require even larger incisions to ensure the appropriate angle in the vertebrae can be achieved by the surgeon.

### BRIEF SUMMARY OF THE INVENTION

In all cases of spinal deformations in which surgery is advised, procedures are generally carried out through large incisions, either in the patient's back (as in a posterior approach), chest or abdomen (as in an anterior approach), or both (as in a combined anterior and posterior approach). Incisions are large enough to allow a surgeon's hands and tools into the affected area. Muscles and ligaments encasing the spine must also be transected and moved. These types of surgeries are generally highly invasive. Recovery from these types of surgeries can often take six months or more, and require extensive physical therapy to strengthen the damaged muscles and potentially ligaments and carry large risk of infection.

The nature of spinal surgeries is also such that a surgeon is often very close to nerves with surgical tools and with limited visibility. This can increase the risk of nerve damage during surgery. Because of the exaggerated size of the incision required, there is also a risk of organ damage during surgery, particularly in anterior approaches.

Advancements in technology, such as the accessibility of imaging procedures, has spurred the development of alternative approaches to traditional surgeries in an attempt to address the challenges posed by conventional approaches. Minimally invasive surgeries, or MIS, are an example of approaches that make heavy use of imaging procedures to guide instrumentation through a patient's body. This substantially reduces the size of the incision required for surgery, causing far less tissue damage.

Existing MIS surgeries have been successful in treating several spinal deformations. These surgeries are critically dependent on the tools and angle of approach. For example, WO Patent Publication No. 2004/049915 to Assel, et al. (the '915 reference), the contents of which are incorporated in their entirety into this disclosure by reference herein. The '915 reference discloses a procedure and related apparatuses that fuse two vertebrae together by driving a two-part implant into the vertebral body, known as AxiaLIF. The implant is first driven into the vertebral bodies of both the L5 and S1. The top of the implant is then extended to distract the two vertebrae along the axis through which the implant was inserted. Because of the position of these two vertebral bodies, a presacral approach is typically required for this surgery.

Other examples include procedures and apparatuses associated with the placement of pedicle screws or facet screws at one level of the spine connected by rods with pedicle screws or facet screws at adjacent spinal levels. The pedicles are bony processes that connect the cylindrical vertebral body anteriorly to the bony processes posteriorly, such as the spinous process and lateral processes. On its posterior face, a vertebra is affixed to its superior neighboring vertebra through a pair of superior facet joints on the superior articular processes. In the inferior direction, it is connected through a pair of facet joints on the transverse process. On its anterior face, a vertebra joins its neighbors by way of intervertebral discs in both directions. The pedicles form the lateral walls of the canal through with the spinal nerves run.

In this example, screws are driven into the pedicles of the vertebrae from the posterior direction and used to adjoin neighboring vertebrae by way of a metal rod between them. Two or more tandem vertebrae are therefore adjoined to one another on each side by metal rods anchored into their pedicles. This is an MIS example of the approach, though the approach can be used with other, non-MIS techniques as well. This example is one that has been successful, but requires extensive hardware, including pedicle screws, metal rods properly shaped to a spine (which requires shaping hardware), and set screws for each pedicle, all of which project outward in the posterior direction. Further, because the strength of this treatment is in aligning vertebrae laterally, it has limited ability to treat deformities in the anterior-posterior axis, such as spondylolisthesis.

Certain existing technologies surround the use of fixation devices for the fusion of adjacent vertebral bodies. Examples of such procedures and associated devices and tools are disclosed in additional patents including U.S. Pat. No. 8,328,847 to Ainsworth et al., filed Aug. 6, 2010, and U.S. Pat. No. 8,292,928 to Cragg et al., filed Sep. 16, 2009, the contents of each of which are incorporated in their entirety into this disclosure by reference herein.

Certain existing technologies surround the methods and apparatus for performing therapeutic procedures in the spine in association with intervertebral spaces. Examples of such methods and apparatus are disclosed in additional patents including U.S. Pat. No. 8,709,087 to Cragg, filed Sep. 12, 2012 and U.S. Pat. No. 6,558,390 to Cragg, filed Feb. 13, 2001, the contents of each of which are incorporated in their entirety into this disclosure by reference herein.

Certain existing technologies surround methods and apparatus for the preparation of a disc space for fusion purposes. Examples of such methods and apparatus are disclosed in additional patents including U.S. Pat. No. 7,799,033 to Assell et al., filed Nov. 19, 2007, U.S. Pat. No. 8,052,613 to Assell et al., filed Oct. 22, 2004, U.S. Pat. No. 7,500,977 to Assell et al., filed Oct. 22, 2004, U.S. Pat. No. 7,914,535 to Assell et al., filed Feb. 6, 2009, and U.S. Pat. No. 8,696,672 to Barnhouse et al., filed Jul. 28, 2011, the contents of each of which are incorporated in their entirety into this disclosure by reference herein.

Certain existing technologies surround the surgical approach to a disc space through soft tissue. An example of such procedures and associated devices and tools are disclosure in additional U.S. Pat. No. 7,763,025 to Assell et al., filed Mar. 13, 2008, the contents of each of which are incorporated in their entirety into this disclosure by reference herein.

Described herein is a process for fusing two adjacent vertebrae. The process described herein makes use of a trajectory through a pedicle of a vertebral body in such a way as to allow the vertebral body to be adjoined to a nearby vertebral body without exterior elements, such as a bridging rod. The process involves a planned trajectory that minimizes potential nerve contact, and allows potential fusion to occur in a disc space not adjacent to nerve or other soft tissue. The process minimizes potential soft tissue damage while still allowing for vertebral body fusion.

### BRIEF DESCRIPTION OF SEVERAL VIEWS OF THE DRAWINGS

FIG. 1—Right lateral view of a spinal section detailing the relevant anatomy

FIG. 2—Right lateral view of a spinal section indicating the inferior-to-superior trajectory 100 into the vertebral column. Lₙ and the nerve root between Lₙ₋₁ and Lₙ₋₂ have been omitted for clarity.

FIG. 3—Inferior view of Lₙ₋₁, looking axially. The line indicating trajectory is continued anterolaterally beyond the vertebral column to help indicate the angle of approach.

FIG. 4—Posterior view of spinal section indicating inferior-to-superior trajectory.

FIG. 5—Flowchart describing a single inferior to superior trajectory example embodiment. This flowchart makes use of certain embodiments, such as a trephine needle, that are intended to provide concrete examples of process implementation, although the exact implementation of these embodiments in practice may vary.

FIG. 6—Anterior, perspective or view of Lₙ₋₁ and Lₙ₋₂ indicating bilateral trajectories.

FIG. 7—Right lateral view of a spinal section indicating the superior-to-inferior trajectory into the vertebral column. Nerve root between Lₙ₋₁ and Lₙ₋₂ has been omitted for clarity.

FIG. 8—Posterior and slightly inferior view of superior-to-inferior trajectory. Imaginary line representing the route has been extended beyond the intended point of termination to assist in alignment.

FIG. 9—Anterolateral view of spinal segment with spondylolisthesis.

FIG. 10—Example embodiment of AxiaLIF-like implant.

### DETAILED DESCRIPTION

Descriptions of certain embodiments serve as examples and do not encompass the entirety of all possible manifestations of the invention. One skilled in the art appreciates that multiple variations of the embodiments enclosed herein may encompass the same inventive concept.

Embodiments are principally concerned with addressing severe spinal deformations by using minimal hardware and causing minimal amount of trauma to a patient. This is accomplished through the use of a specific path through the patient's vertebral column, either in an inferior-to-superior trajectory 100 or superior-to-inferior trajectory 112, and an implant device.

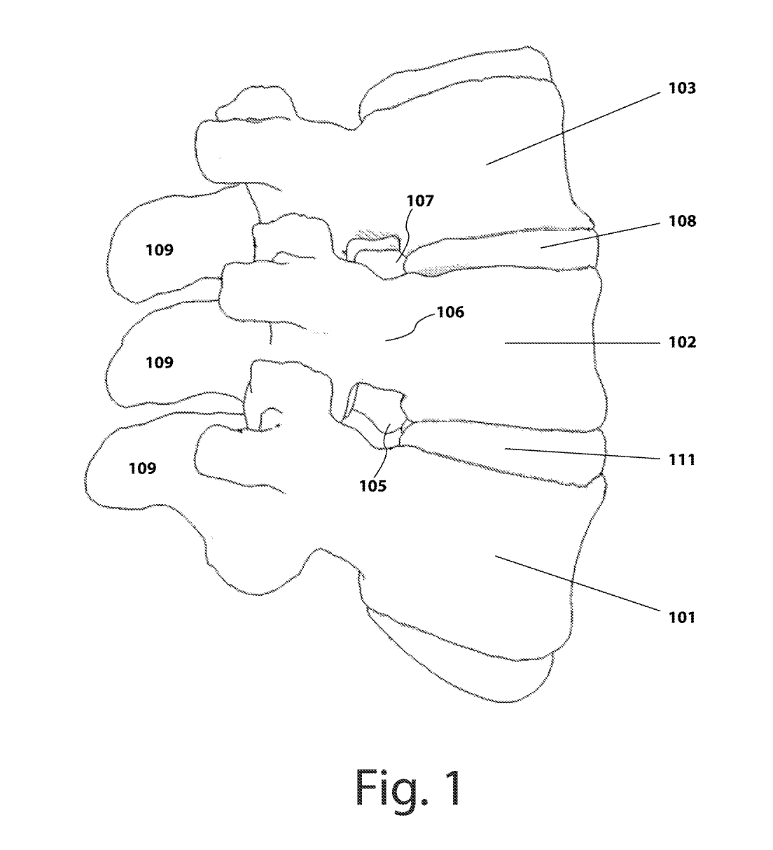

For the purposes of description in these embodiments, a reference lumbar vertebra appropriate for a given surgery is referred to as Lₙ 101, as seen in FIG. 1. Other vertebrae reference this position and decrement the number, indicating cranial direction from Lₙ 101. For example, if Lₙ 101 refers lumbar vertebra 4 (L4) in a particular patient, then Lₙ₋₁ 102 refers to L3, and Lₙ₋₂ 103 refers to L2. The “L” notation is not intended to convey a confinement of the embodiment to only lumbar vertebrae. For example, in the event that Lₙ 101 refers to L2, Lₙ₋₂ 103 would then refer to a thoracic vertebra. The Lₙ₋₂ 103 notation is still retained in such a case. In the event that the procedure traverses the L5-S1 joint, the surgeon may also be required to drill holes through one ilium or both ilia to access the joint.

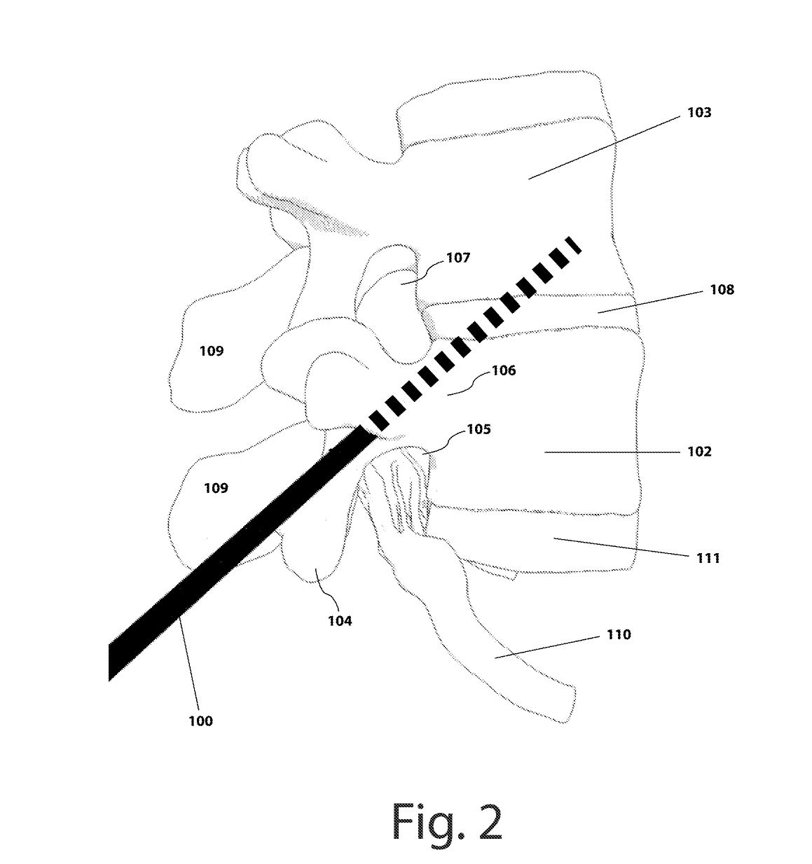

In an embodiment shown in FIG. 2, the approach 100 through the spine begins posterior, inferior, and slightly lateral to Lₙ₋₁ 102, just posterior to Lₙ 101. The approach trajectory then enters the pedicle 106 of Lₙ₋₁ 102 towards Lₙ₋₂ 103, at approximately 45 degrees off of the coronal plane, as seen in FIG. 2. In embodiments of the invention, the angle of this approach trajectory is such that an implant with an 18 mm diameter placed through an aperture along the approach path will continue through contiguous pedicle bone, and not breach the superior or inferior faces of the pedicle once inside. Thus, the diameter of approach trajectory in embodiments of the invention does not exceed 10 mm. In the preferred embodiment of the invention, the diameter of the approach is 9 mm or less to accommodate an generally cylindrical-shaped implant having a diameter of 9 mm or less.

Certain embodiments will make use of an imaging technique 113 to facilitate identification of the proper path through the patient. Such embodiments ensure accurate trajectories and minimize the possibility of nerve contact. In such embodiments, the imaging technique 113 may take the form of an intra-operative “O-arm Surgical Imaging System” (“O-arm”), which provides real time, multi-dimensional imaging. In alternative embodiments, the imaging technique 113 may instead take the form of fluoroscopy or X-ray.

The approach continues in a straight line above the neural foramen 105 formed between Lₙ 101 and Lₙ₋₁ 102, as seen in FIG. 2. The angle of approach is such that, once inside, the remainder of the route continues to traverse contiguous bone without exiting so as to avoid nerve contact, such as with the Lₙ₋₁ 102 nerve root 110. The approach therefore continues into the vertebral body of Lₙ₋₁ 102 beneath the neural foramen 107 formed between Lₙ₋₁ 102 and Lₙ₋₂ 103.

The approach continues through the intervertebral disc 108 between Lₙ₋₁ 102 and Lₙ₋₂ 103, and finally into the vertebral body of Lₙ₋₂ 103, where the route terminates.

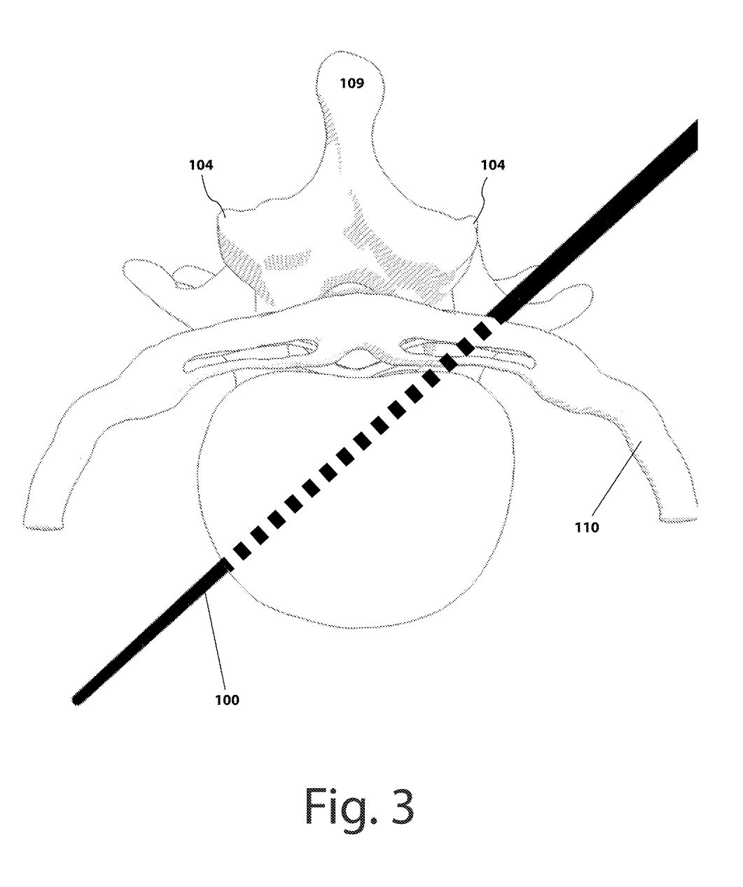

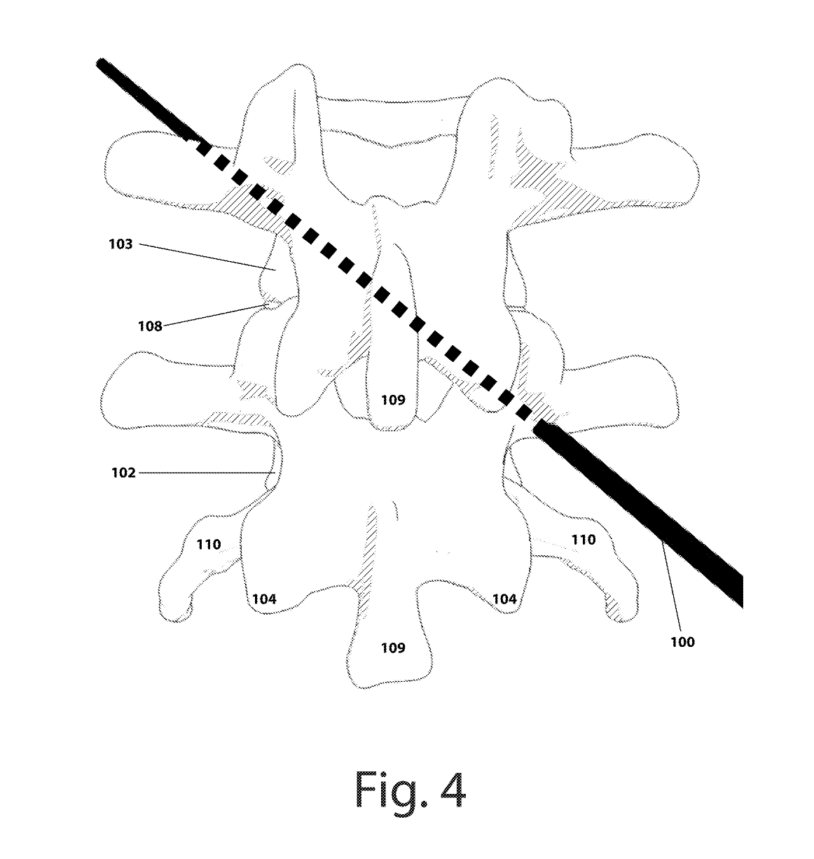

The angle of the approach 100 begins laterally to the inferior facet joint 104 of Lₙ 101 and Lₙ₋₁ 102, and is directed medially as it progresses, as seen in FIG. 3. The trajectory aims for the portion below the facet joint between Lₙ₋₂ 103 and Lₙ₋₃ when regarded posteriorly along anterior-posterior axis, as seen in FIG. 4. Per this trajectory, the route should already be medial to the foramen formed between Lₙ₋₁ 102 and Lₙ₋₂ 103 once it penetrates Lₙ₋₂ 103. The angle is such that if an imaginary line were to continue the route beyond the vertebral body of Lₙ₋₂ 103, that line would exit Lₙ₋₂ 103 contralaterally, approximately midway up the vertebral body and lateral to the midline. This angle is approximately 48.5 degrees off of the sagittal plane. This approach can be bilateral, mirroring this route on either side of the spinous processes 109.

Landmarks on skin will vary depending on vertebral anatomy of the individual and angles for safe passage, but generally will be about 6 inches lateral to midline and 6 inches caudal to Lₙ₋₁ 102. This “inferior to superior” embodiment is preferred for operations wherein L4 and L5, L3 and L4, and/or L2 and L3 require fusion.

In an embodiment, a rigid, pilot implement, such as a trephine needle is maneuvered through this trajectory by a surgeon with the aid of an imaging technique 113, such as an intra-operative O-arm. The pilot implement is long enough to penetrate the full depth of the approach while still being exposed outside of the patient's back, and durable enough to bore the pilot hole through vertebral bone. In the event that a trephine needle is used as the pilot implement, the top of the needle is removed once the needle is in the appropriate position, leaving a cannulated center. The hollow remainder of the needle acts as a canal, through which a guiding implement, such as a guide wire, is threaded. The guiding implement need not be hollow, but is long and thin, allowing other cannulated tools to follow the path demarcated by the guiding implement by sliding along its length. An implant, such as the one described in the '915 reference, for example, can then be guided down the trajectory over a guiding implement such as a guide wire in this way.

The implant follows the trajectory established by a pilot implement such as a trephine needle by following the guiding implement. The implant therefore traces the same steps through bone while avoiding nerve contact. The final position of the implant is determined when the distal portion of the implant is lodged in Lₙ₋₂ 103, and the proximal portion of the implant is lodged in Lₙ₋₁ 102. The adjoining portion of the implant, such as a spanning distraction portion, is positioned in the intervertebral disc 108 between Lₙ₋₁ 102 and Lₙ₋₂ 103.

Once the implant is in the appropriate position and is bridging two vertebrae, the distal portion of the implant can be adjusted at the surgeon's discretion. Because the implant threads are embedded in vertebral bone, adjustment of the distance between the distal portion and proximal portion of the implant can articulate the vertebrae relative to one another. This process of distraction therefore articulates the second vertebra relative to the first, such as between Lₙ₋₁ 102 and Lₙ₋₂ 103.

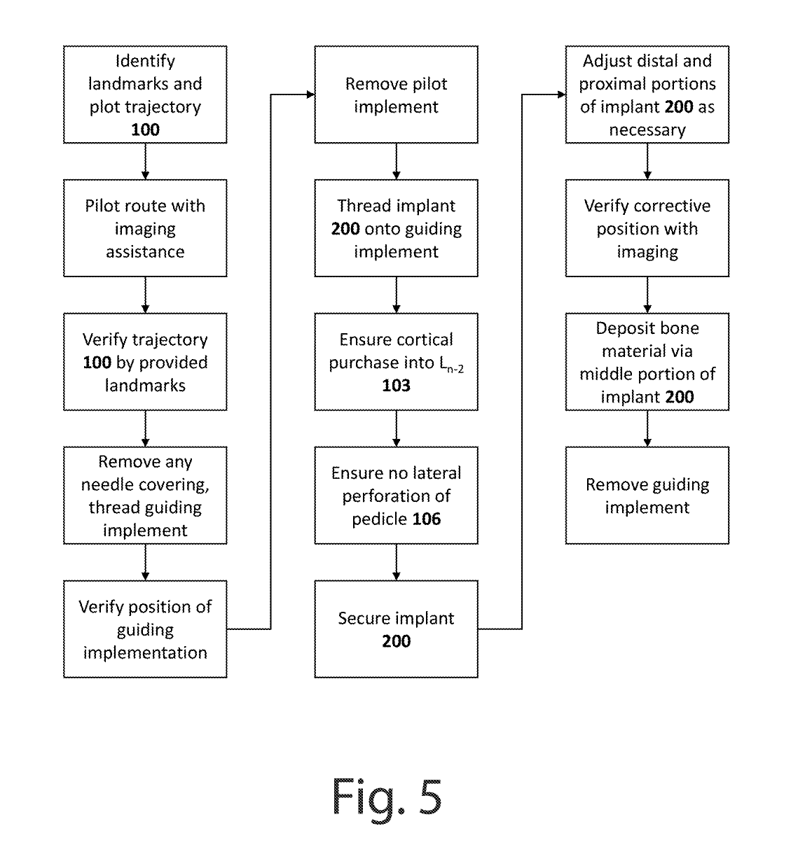

In an embodiment, the hollow, adjoining portion of the implant, such as a spanning distraction portion, includes multiple, laterally bored holes. Such an embodiment is described in FIG. 5. Bone or bone-like material, including allograft or autograft bone or similar substances, for example cancellous bone, bone chips, durable bone matrix, bone morphogenic protein or other substances or any combination thereof, can therefore be added to the implant and/or placed within the disc space, and will be deposited at its position in the intervertebral disc space 108 between Lₙ₋₁ 102 and Lₙ₋₂ 103. Addition of bone material further strengthens the fusion and does not require additional metal, such as spanning distraction portions. The present inventor has observed that use of bone or bone-like material over additional hardware in this context mitigates undesirable forces on the vertebral bodies.

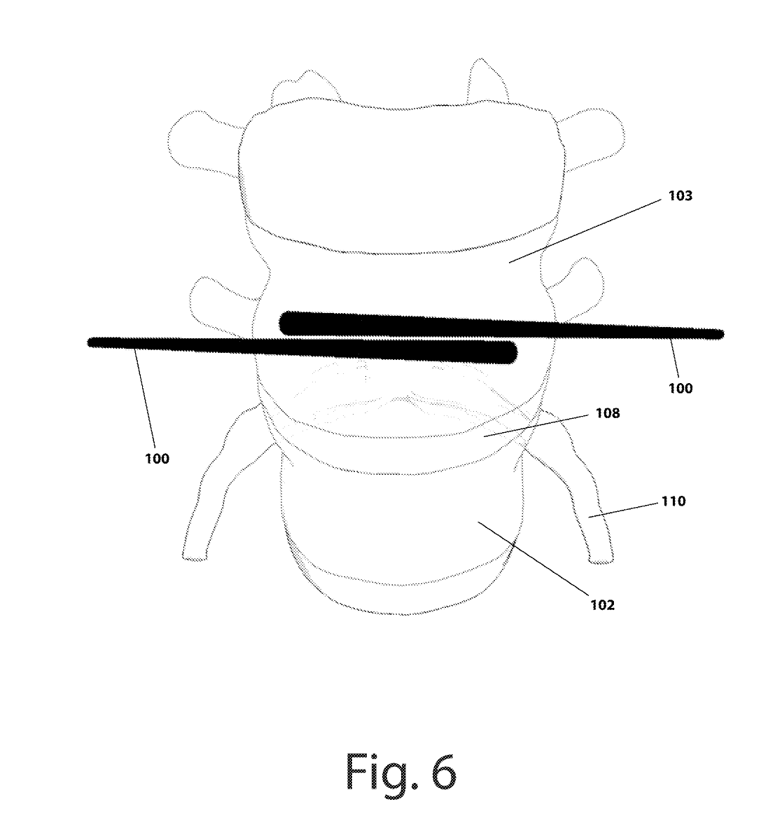

The same approach within a reasonable margin is performed on the contralateral side. In one embodiment, two implants are embedded into the same two vertebrae, following approximately the same trajectory 100, but providing a narrow margin between one another, as seen in FIG. 6. Each implant approaches the vertebral joint from a different axis, mirrored across the midline. Because two posterior-lateral approaches from two different axes are used in combination, two points of articulation are provided by this procedure.

In embodiments of the invention the efficacy of the fusion depends heavily on the strength of the fusion. This approach therefore maximizes cortical purchase through the vertebrae, but does not require deep penetration into cancellous bone. One major advantage of the process associated with the preferred embodiment of the invention is that it provides high strength in this way without requiring adjoining rods. While rods provide reinforced support, they also translate forces between vertebrae. Because of this translation, the rods tend to loosen over time as the vertebrae try to move relative to each other. By eliminating this intermediate joint, the embodiments presented here reduce the loosening effect, requiring fewer subsequent surgeries for maintenance, and providing a strong, lasting fusion.

Another major advantage of these embodiments is demonstrated in the potential for easy removal. While other MIS operations cause relatively little trauma when compared to more traditional approaches during implantation, there is no inherent extraction mechanism in the event that the hardware needs to be removed. These operations therefore may require invasive surgeries for removal, causing heavy tissue damage. By contrast, the embodiments presented herein are easily extracted by the same route through which they were implanted, by unscrewing the implanted hardware. As with implantation, the entire removal process can be accomplished percutaneously.

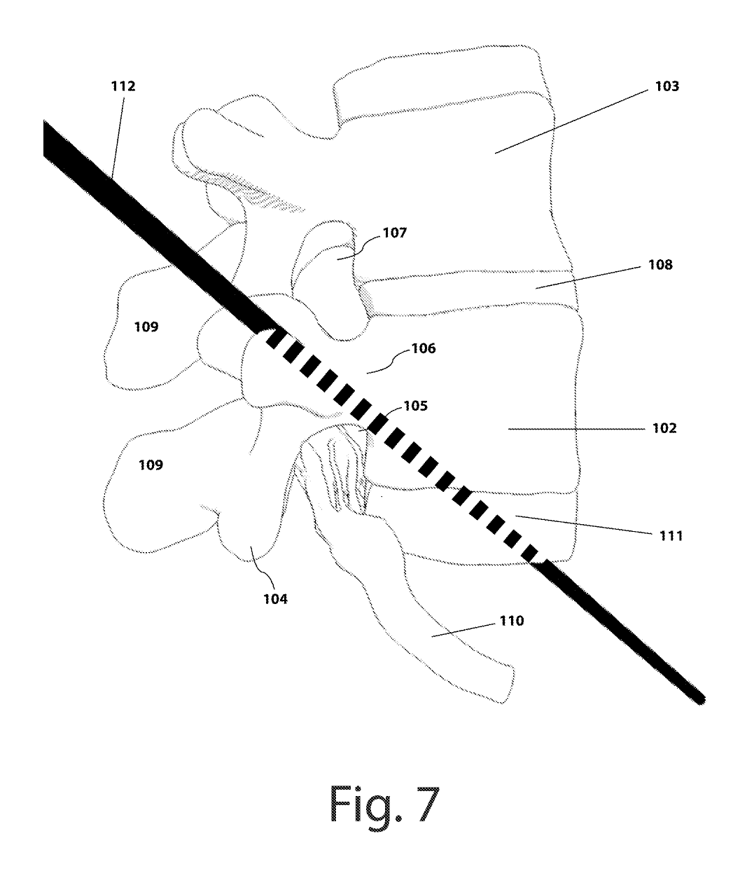

In certain situations, a surgeon may determine that the superior-to-inferior trajectory 112 hereby presented as an alternative embodiment of the invention is more appropriate, rather than the inferior-to-superior one already described herein. In such situations, the central principle of the embodiments presented here can be preserved through an alternative route, as demonstrated in FIG. 7.

In this embodiment, the route begins superior and lateral to Lₙ₋₂ 103. The route 112 takes a steep angle towards Lₙ₋₁ 102 of about 47 degrees off of the coronal plane, and about 48 degrees off of the sagittal plane. The route enters the pedicle of Lₙ₋₁ 102 on the superior face, as seen in FIG. 7, and continues through the pedicle internally, running along the superior/anterior face of the pedicle and into the vertebral body of Lₙ₋₁ 102. Slight modifications made to this trajectory by the surgeon as necessary may briefly expose the route at the inferior/medial face of the pedicle. Because of the downward trajectory of the nerve roots 110 from the foramen, there are several millimeters of tolerance between the nerve root and the pedicle at this position, however, allowing ample clearance in the event that it is deemed necessary by the surgeon.

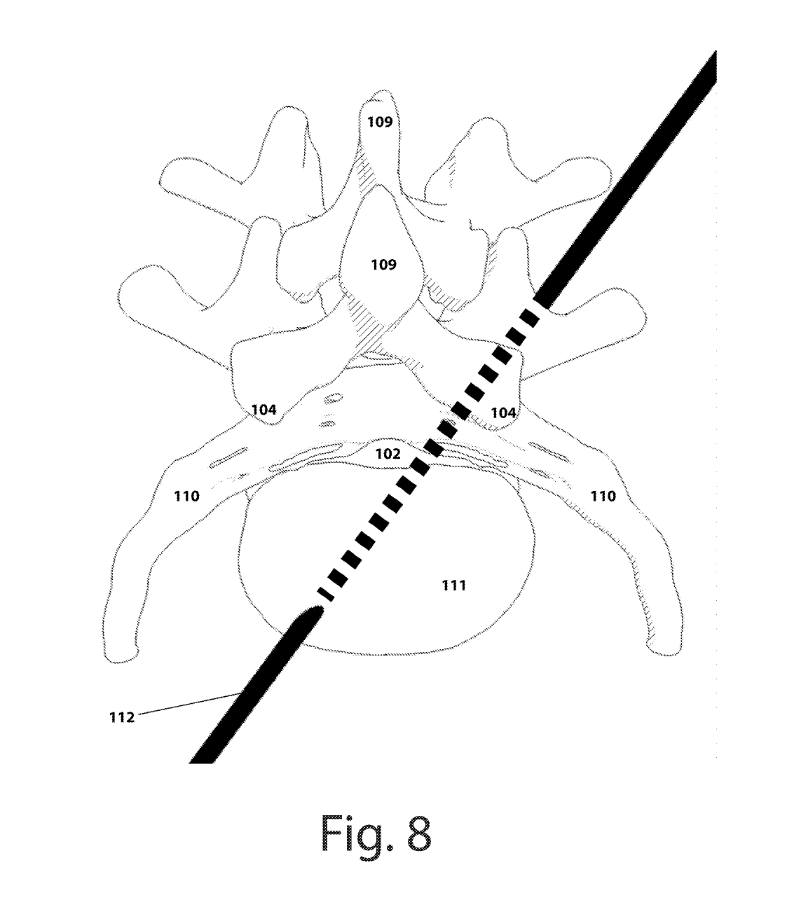

The route continues downward through the intervertebral disc 111 between Lₙ₋₁ 102 and Lₙ 101, as seen in FIG. 8. The route then advances into the vertebral body of Lₙ 101, where it terminates. The route is such that an imaginary line continuing the route would exit Lₙ 101 anterolaterally, a few millimeters below the top of the vertebral body. The trajectory of this route may be preferred by certain surgeons in some cases of L4/L5 or L5/S1 fusions.

The embodiments presented here may be useful in where in distraction for a collapsed disc space is required on one side, but compression is called for on the other side. By offering several articulating points of contact, this process can adjust the two sides of one vertebral body face relative to one another.

These embodiments can be used in conjunction with other fusion mechanism, the '915 reference for example, which discloses a facet screw fusion system. One advantage of these embodiments when used in this way is a superior removal process. In the event that a surgeon opts for implant removal, the same route can be retraced, allowing the surgeon to simply loosen the implant for removal along the same, minimally invasive path. This is in contrast to existing solutions, which require full surgeries to remove extensive hardware, even MIS procedures that cause little tissue damage on implantation.

Another example of an application that may make use of a combination with other fusion mechanisms is an application in conjunction with spanning distraction rods. Any of these embodiments could be used to complement thoracic surgeries, such as a T10-S1 spanning distraction rod, for example. Alternative embodiments are not limited to lumbar vertebrae, but may be incorporated elsewhere, such as in thoracic vertebral fusions, for example.

While it is possible to use these embodiments with additional hardware, an advantage of these embodiments is the ability to forgo additional hardware, such as spanning distraction rods. These embodiments can, instead, include bone or bone like material deposited into any intervertebral disc, such as 108 for an inferior-to-superior approach, or 109 for a superior-to-inferior approach. The present inventor has noted that the strategy associated with the inventive concept disclosed herein has the advantage of reducing the likelihood of trauma on a patient, by reducing forces applied to the body.

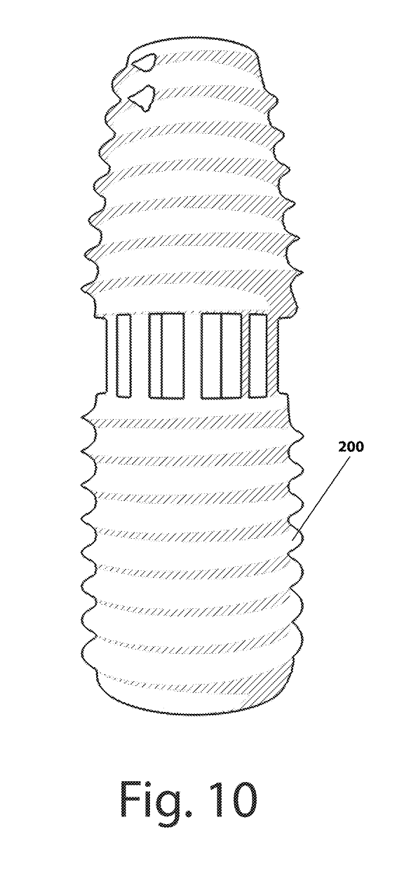

Another advantage of these embodiments is that the single trajectory from the posterior, lateral approach, through the pedicle and into the vertebral body more efficiently treats spinal deformations, such as spondylolisthesis, due to its approach through three axes relative to the spine. The trajectory 100 taken by a surgeon in embodiments presented here extend farther across the intervertebral space between the two displaced vertebrae than conventional techniques. This trajectory 100 therefore provides a surgeon with a greater degree of leverage for addressing such deformations. A bilateral approach provides even more flexibility along these axes. This is because the bilateral approach allows differential tightening at two points of contact, allowing a surgeon to adjust the relative positions of two adjacent vertebrae in three dimensions by simply adjusting the relative contraction/distraction of the two implants. In embodiments of the invention, the implant may comprise substantially cylindrical threaded implants with a middle piece designed to distract two opposing components that are threaded into a superior and inferior vertebral body. In embodiments of the implant, the implant comprises an AxiaLIF-like implant illustrated in FIG. 10, though of a smaller diameter than the AxiaLIF implant known in the art, with an outer dimension suited to a bore through a pedicle. The general components of such a device may include a distal portion, which is intended to become embedded in Lₙ₋₂ 103 in the inferior-to-superior trajectory 100, a middle portion that will be embedded in an intervertebral disc space, such as disc space 108 between Lₙ₋₁ 102 and Lₙ₋₂ 103, and a proximal portion, which is intended to become embedded in Lₙ₋₁ 102 in the inferior-to-superior trajectory 100. In embodiments of the invention, the implant is adapted from the pre-sacral approach to the approach contemplated herein and so shaped such that its diameter is no more than 10 millimeters, and in alternative embodiments the diameter is specifically 8 millimeters or 9 millimeters to correspond with the size of a bore through a pedicle that does not compromise the integrity of a pedicle. The use of such an implant effectively gives a surgeon two points of contact between the same two vertebrae, and therefore affords a greater degree of freedom. This not only allows the correction of conditions previously very difficult to treat, but also provides far greater precision.

An important advantage of these embodiments is that they provide two adjustable points of contact between any two vertebrae. By slightly modifying the angles and adjusting the degree to which the implants are tightened, the alignment between the two vertebrae can be adjusted in three dimensions. This provides a surgeon with exquisite and powerful tools to modify vertebral alignment that are not provided by other surgical techniques, and with much more precise control over the exact orientation.

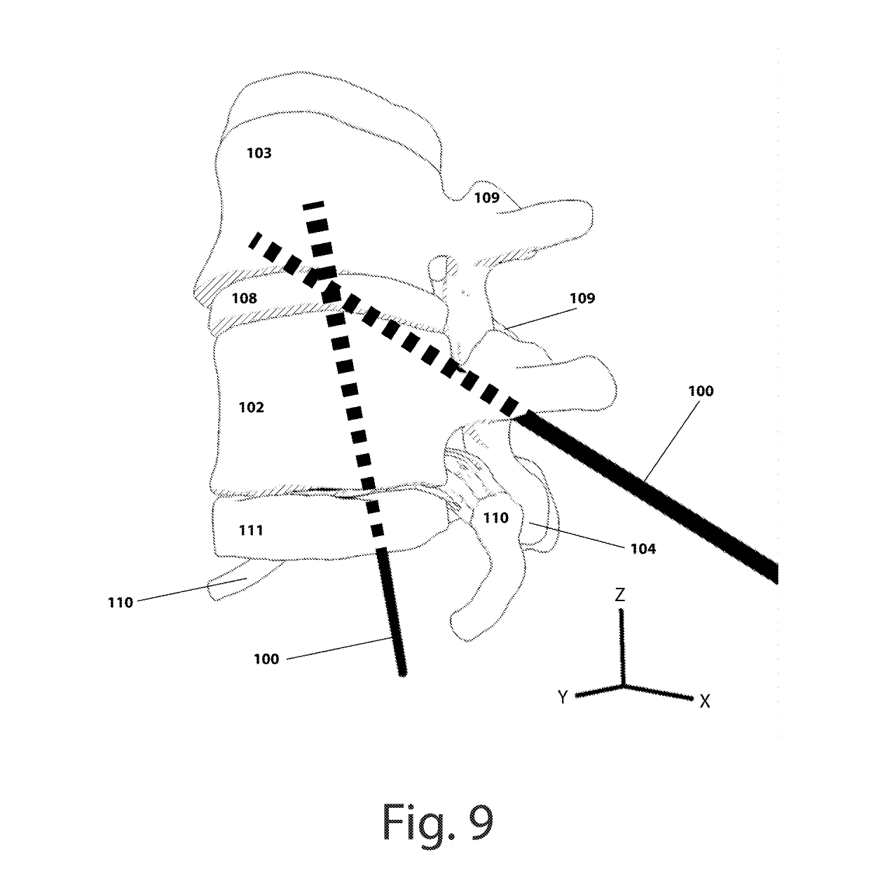

For example, in a case of spondylolisthesis, as seen in FIG. 9, wherein the vertebral body Lₙ₋₂ 103 is positioned anteriorly and rightward relative to the vertebral body of Lₙ₋₁ 102, a greater contraction of a left implant, such as the one depicted in FIG. 10, will provide greater movement posteriorly and laterally. The vertebral body can be more accurately repositioned using these two points of contact, and can be used in conjunction with several other techniques at the surgeon's preference. Referencing FIG. 9 once again, a coordinate system demarcates an X, Y and Z direction. Lₙ₋₂ 103 is displaced anteriorly in the “Y” direction and slightly superiorly in the “Z” direction, with respect to Lₙ₋₁ 102. Bilateral instances of inferior-to-superior trajectory 100 are used, allowing for vertebral body manipulation across all three dimensions of movement.

By providing these multiple adjustable points of contact, the process described herein is particularly well suited for treating spinal deformations that are otherwise very difficult to treat, such as spondylolisthesis. Existing MIS approaches are very effective in their ability to minimize tissue damage and trauma during operation. However, MIS approaches are also very limited in their ability to modify spatial orientation in the anterior-posterior axis, due to the restricted axis of approach. This makes vertebral displacements along any other axis technically very challenging to correct, and often invalidates the use of MIS approaches. To address these problems, the embodiment described herein provides multiple, biaxial entries into vertebrae at specific angles that is able to allow adjustment in the anterior-posterior axis while avoiding nerve contact. This procedure still allows articulation around other axes, and so allows a surgeon to correct a displacement along any or all of these axes, such as a spondylolisthesis, without sacrificing the benefits of an MIS technique.

By using X-ray guided, minimally invasive tools with high precision, surgeons are able to substantially lessen the risk of nerve damage. By following the trajectory disclosed here, surgeons are able to further reduce this risk by staying in bone throughout the entire procedure. The trajectory disclosed here minimizes the possibility of nerve contact, while still maximizing spatial articulation in three dimensions.

In general, certain embodiments of the invention are performed by a medical practitioner, where such medical practitioner may include a number of entities related to a surgical procedure, including but not limited to surgeons, physician's assistants, nurses, technicians, neurodiagnostic technicians, and anesthesiologists. Certain embodiments of the invention are performed in conjunction with a number of instruments, including, but not limited to imaging 113 or scanning devices such as, for example, biplanar fluoroscopes (also referred to as C-Arm fluoroscopes). Such imaging 113 or scanning devices captures images of a patient through various views, including but not limited to a lateral view, an oblique view, a posterior and anterior-posterior (AP) view, superior view, and distal views of the patient. Such imaging 113 or scanning devices are used in portions, or throughout therapeutic procedures described in certain embodiments of the invention.

In general, therapeutic approaches in certain embodiments of the invention occurs after a patient undergoes anesthesia, disinfection, and other standard procedures and practices related to surgery and/or spinal surgeries known to persons having ordinary skill in the art. In certain embodiments, a patient is placed under general anesthesia, or optionally remain conscious, and/or otherwise be placed under a general or local analgesic for the duration of the methods and procedures described. In certain embodiments, the therapeutic approach is performed on a patient placed in a prone position.

In the foregoing specification, specific embodiments have been described. However, one of ordinary skill in the art appreciates that various modifications and changes can be made without departing from the scope of the invention as set forth in the claims below. Accordingly, the specification and figures are to be regarded in an illustrative rather than a restrictive sense, and all such modifications are intended to be included within the scope of present teachings.

The benefits, advantages, solutions to problems, and any element(s) that may cause any benefit, advantage, or solution to occur or become more pronounced are not to be construed as a critical, required, or essential features or elements of any or all the claims. The invention is defined solely by the appended claims including any amendments made during the pendency of this application and all equivalents of those claims as issued.

Moreover in this document, relational terms such as first and second, top and bottom, and the like may be used solely to distinguish one entity or action from another entity or action without necessarily requiring or implying any actual such relationship or order between such entities or actions. The terms “comprises,” “comprising,” “has”, “having,” “includes”, “including,” “contains”, “containing” or any other variation thereof, are intended to cover a non-exclusive inclusion, such that a process, method, article, or apparatus that comprises, has, includes, contains a list of elements does not include only those elements but may include other elements not expressly listed or inherent to such process, method, article, or apparatus. An element proceeded by “comprises . . . a”, “has . . . a”, “includes . . . a”, “contains . . . a” does not, without more constraints, preclude the existence of additional identical elements in the process, method, article, or apparatus that comprises, has, includes, contains the element. The terms “a” and “an” are defined as one or more unless explicitly stated otherwise herein. The terms “substantially”, “essentially”, “approximately”, “about” or any other version thereof, are defined as being close to as understood by one of ordinary skill in the art. The terms “coupled” and “linked” as used herein is defined as connected, although not necessarily directly and not necessarily mechanically. A device or structure that is “configured” in a certain way is configured in at least that way, but may also be configured in ways that are not listed. Also, the sequence of steps in a flow diagram or elements in the claims, even when preceded by a letter does not imply or require that sequence.

What is claimed is:

1. A process for fusing two or more adjacent vertebrae utilizing a trajectory that avoids contact with nerve structures by traversing through two or more pedicles comprising the following steps:
identifying the pedicles of two or more vertebrae to be fused and the disc space between said two or more vertebrae;
planning a route through at least one pedicle to terminate in an identified disc space;
creating access along a trajectory following said route;
inserting an implant into said identified disc space following said trajectory;
filling the intervertebral disc space with bone material to promote fusion by placing said bone material through the trajectory and into said intervertebral disc space.

2. The process of claim 1, further comprising the steps of
identifying a trajectory to create a pathway with a diameter no larger than 9 millimeters that intersects a first vertebra from a starting position lateral to a midsagittal plane of the first vertebra, enters the first vertebra at a pedicle, and continues along a path approximately 47-degrees off of a coronal plane and 48 degrees off of a sagittal plane towards the midsagittal plane of a second vertebra, passing above a neural foramen formed between the first vertebra and the second vertebra and traversing an intervertebral disc space separating them there between, in conjunction with an imaging system;
inserting a trephine needle along said trajectory, which, with assistance from the imaging system, bores the initial cavity through said trajectory; and
threading of a guide wire through the trephine needle to demarcate the trajectory for other tools.
